(19) United States
(12) Patent Application Publication
(10) Pub. No.: US 2023/0418264 A1
(43) Pub. Date: Dec. 28, 2023

(54) AROMA DISPLAY, AROMA DISPLAY CONTROL DEVICE, AROMA SCHEDULING SERVER AND COMPUTER PROGRAM

(71) Applicant: AROMAJOIN CORPORATION, Kyoto-shi, Kyoto (JP)

(72) Inventors: Aiko YASUI, Kyoto-shi (JP); Dong Wook KIM, Kyoto-shi (JP); Duc Hanh TRAN, Kyoto-shi (JP); Alexander Jeffrey MOSKOWITZ, Kyoto-shi (JP)

(21) Appl. No.: 18/035,814

(22) PCT Filed: Nov. 10, 2021
(86) PCT No.: PCT/JP2021/041259

§ 371 Date: May 8, 2023

(30) Foreign Application Priority Data
Nov. 18, 2020 (JP) 2020-191912

(51) Int. Cl. G05B 19/4155; A61L 9/12

(52) U.S. Cl. CPC G05B 19/4155; A61L 9/12; G05B 2219/40576; A61L 2209/133; A61L 2209/11

(57) ABSTRACT

An aroma display that enables, by an easy operation, generation of scents fit for situations, at various scenes and times includes: a message receiving unit 180 for receiving an external message; a correspondence storage means for storing correspondence between an event name and aroma information related to a scent to be generated at the event; an event information extracting means for extracting event information including an event name and time information related to the event from the message received by the message receiving unit 180; aroma information reading units 185 and 186 for reading aroma information corresponding to the event name from the correspondence storage means; and an aroma generating unit 194 generating a scent in accordance with the aroma information read by aroma information reading units 185 and 186.

### TECHNICAL FIELD

The present invention relates to an aroma display and, more specifically, to an aroma scheduling server for distributing scents in accordance with a schedule using an aroma display on which a plurality of aroma cartridges is loaded, to allow emission of various scents, an aroma display device and an aroma display control device as well as a computer program therefor. The present application claims convention priority on Japanese Patent Application No. 2020-191912 filed on Nov. 18, 2020, and incorporates the description of this Japanese Application in its entirety.

### BACKGROUND ART

Human communication is done in various modalities based on human senses. Most frequently used are visual and auditory communications. By contrast, olfaction or sense of smell, on which we rely considerably in our lives, is hardly used in communication. However, if the sense of smell can be utilized in addition to visual and auditory senses, communication would be more efficient and different people would be able to share their experiences more profoundly.

Communication is not the only field in which an aroma or a scent plays a significant role. By way of example, mediation would be more satisfactory if surrounded by a calming and relaxing scent. After working for hours, a brisk, refreshing aroma would be perfect for a break time.

To meet this demand, a device that generates scents in accordance with instructions (hereinafter referred to as an “aroma display”) as described, for example, in Patent Literature 1 has been developed and come to be used widely.

### CITATION LIST

### Patent Literature

Patent Literature 1: JP2014-092673A

### SUMMARY OF INVENTION

### Technical Problem

In our daily life, however, it is troublesome to operate an aroma display to generate desired scents from one situation to another. Therefore, there is a demand for an aroma display, an aroma display control device and a scheduling server that enable by easy generation of scents fit for situations, at various scenes and times in daily life.

### Solution to Problem

According to a first aspect, the present invention provides an aroma display, including: a message receiving means for receiving an external message; a correspondence storage means for storing correspondence between an event name of an event and aroma information related to a scent to be generated at the event; an event notification extracting means for extracting an event notification including an event name from a message received by the message receiving means; an aroma information reading means for reading aroma information corresponding to the event name from the correspondence storage means; and an aroma emitting means for emitting a scent in accordance with the aroma information read by the aroma information reading means.

According to a second aspect, the present invention provides an aroma display, including: a timer; a correspondence storage means for storing correspondence between an event name of an event and aroma information related to a scent to be generated at the event; a schedule storage means for storing event information including an event name of an event to be scheduled and time information related to the event; a schedule extracting means for extracting an event based on time measured by the timer and on the time information of each piece of the event information stored in the schedule storage means; and an aroma emitting means for emitting a scent in accordance with the aroma information corresponding to the event name included in the event information of the event extracted by the schedule extracting means, in accordance with the correspondence stored in the correspondence storage means.

Preferably, the aroma emitting means allows loading of a plurality of aroma cartridges each having an identification number indicative of its scent source, and is capable of emitting a scent from any designated aroma cartridge among the plurality of aroma cartridges; the aroma information includes the identification number. The aroma emitting means includes: an identification number storage means for storing the identification number allotted to each of the plurality of aroma cartridges loaded to the aroma emitting means; and a cartridge driving means for driving that aroma cartridge which has the identification number included in the aroma information among the plurality of aroma cartridges loaded to the aroma emitting means and thereby for generating a scent.

More preferably, the event notification may include a duration of scent emission; and in response to the event notification including information related to the duration of scent emission, the cartridge driving means drives the aroma cartridge for the duration from the start time of the event.

Further preferably, in the aroma display, the time information of the event information further includes an event end time; and the cartridge driving means drives the cartridge driving means from the event start time until the event end time.

According to a third aspect, the present invention provides an aroma display control device, for controlling one or more aroma displays, including: a communication destination information storage means for storing communication destination information enabling communication with the one or more the aroma displays as communication destinations; a message receiving means for receiving an external message; a correspondence storage means for storing correspondence between an event name of an event and aroma information related to a scent to be generated at the event; an event notification extracting means for extracting an event notification including an event name from a message received by the message receiving means; an aroma reading means for reading aroma information corresponding to the event name from the correspondence storage means; and a command transmitting means for transmitting a command to an aroma display as a communication destination stored in the communication destination information storage means so that a scent is emitted in accordance with the aroma information read by the aroma reading means.

Preferably, the command transmitting means includes a full transmission means for transmitting the command to each of the aroma displays of communication destination stored by the communication destination information storage means to generate a scent in accordance with the aroma information read by the aroma reading means.

More preferably, the event notification may include information related to a duration of scent emission in an event indicated by the event notification; and the command transmitting means includes a means, responsive to the event notification including the information related to the duration, for transmitting the command including the event name and the information related to the duration, to an aroma display of communication destination stored by the communication destination information storage means to generate a scent in accordance with the aroma information read by the aroma reading means.

According to a fourth aspect, the present invention provides a computer program causing a computer to function as any of the above-described devices.

According to a fifth aspect, the present invention provides an aroma scheduling server, including: a timer; a correspondence storage means for storing correspondence between an event name of an event and an identification number of a scent to be generated at the event; an access information storage means storing access information to an aroma display; a schedule information storage means, receiving from a user, inputs of an event name, start time and end time of the event, and location information specifying the location where the event takes place, for storing schedule information including the event name, the start time and the end time, and the location information; an identification number reading means for reading, from the correspondence storage means, an identification number of a scent corresponding to the event name input by the user, and for adding it to the schedule information corresponding to the event name; a schedule information reading means for detecting whether a piece of schedule information having substantially the same start time as the time indicated by the timer exists in the schedule information stored in the schedule information storage means, and for reading the detected piece of schedule information; and an event notification transmitting means for transmitting an event notification including scent identification information, scent emission duration, and an event name determined in accordance with the read piece of schedule information to a destination determined by the access information included in the piece of schedule information read by the schedule information reading means.

Preferably, the schedule information storage means includes an emission-interval-added schedule information storage means, receiving, from a user, inputs of an event name, start time and end time of the event, and location information specifying the location where the event takes place and time interval for emitting a scent at the event, for storing schedule information including the event name, the start time and the end time, the location information and the emission time interval; the scheduling server further includes: an aroma emission time calculating means for calculating aroma emission time at which the scent is to be emitted for the event based on the event start time and the emission time interval; the schedule information reading means includes an aroma emission schedule information reading means for detecting whether a piece of schedule information having the emission time substantially the same as the time indicated by the timer exists in the schedule information stored in the emission-interval-added schedule information storage means, and for reading the detected piece of schedule information.

According to a sixth aspect, the present invention provides a computer program causing a computer to function as any of the above-described scheduling servers.

The foregoing and other objects, features, aspects and advantages of the present invention will become more apparent from the following detailed description of the present invention when taken in conjunction with the accompanying drawings.

### BRIEF DESCRIPTION OF DRAWINGS

FIG. 1 is a block diagram schematically showing a configuration of an aroma scheduling system in accordance with a first embodiment of the present invention.

FIG. 2 is a block diagram schematically showing a configuration of an aroma display shown in FIG. 1.

FIG. 3 is a flowchart showing a control structure of a program executed by a control processor of the aroma display shown in FIG. 2.

FIG. 4 is a block diagram schematically showing a configuration of a panel-type computer shown in FIG. 1.

FIG. 5 is a flowchart showing a control structure of a program executed by the panel-type computer shown in FIG. 1.

FIG. 6 is a block diagram schematically showing a configuration of an in-company scheduling system in accordance with a second embodiment of the present invention.

FIG. 7 is a block diagram schematically showing a configuration of the in-company scheduling server shown in FIG. 6.

FIG. 8 is a schematic diagram showing relations of programs and data stored in the in-company scheduling server shown in FIG. 7.

FIG. 9 shows contents of a schedule table shown in FIG. 8.

FIG. 10 shows a table configuration of a facility master table shown in FIG. 8.

FIG. 11 shows a table configuration of an event master table shown in FIG. 8.

FIG. 12 shows a table configuration of an aroma master table shown in FIG. 8.

FIG. 13 shows a table configuration of an aroma display master table shown in FIG. 8.

FIG. 14 shows a sequence when a schedule is to be registered from a computer to the in-company scheduling server in accordance with a second embodiment of the present invention.

FIG. 15 is a flow chart showing a control structure of a program executed by the in-company scheduling server for aroma scheduling in accordance with a second embodiment.

FIG. 16 shows a configuration of a management table of an aroma display as an object of management, held in the panel-type computer shown in FIG. 6.

FIG. 17 is a flowchart showing a control structure of a program for the panel-type computer to drive an aroma display as an object of management, in accordance with the second embodiment.

### DESCRIPTION OF EMBODIMENTS

In the following description and in the drawings, the same components are denoted by the same reference characters. Therefore, detailed description thereof will not be repeated.

### First Embodiment

On the Internet, a so-called scheduling service is widely offered. A scheduling service make it possible to keep records of where and what events take place at any specific time of any specific day. As a result, it is possible to check a schedule of a person, a group or the like, day by day, week by week, month by month and year by year. Further, it often includes a service to send an e-mail (hereinafter simply referred to as a mail) to a specific address of a mail (hereinafter, “a mail address”) notifying the start of an event to a specific mail address at the start time or prescribed time before the start of the event.

These events may include the time for meditation and a work break for refreshment as mentioned above, which may be more satisfactory if specific aroma or scent is generated at the event site. The first embodiment is to enable timely operation of an aroma display installed at a specific location, by utilizing such a scheduling service.

Use of a scheduling service for operating the aroma display in this manner faces a problem that the operation of scheduling service itself cannot be customized. A typical scheduling service allows registration of event name, event start time, end time (or event duration), place, participant name and participant contact information only, and it is difficult to designate a scent.

Therefore, in the embodiment as described in the following, combinations of an event name and a scent to be generated for the event are stored beforehand in the aroma display. The event is registered with the scheduling service, and at the event start time, a mail designating the event name is sent to the aroma display set on the event site. Thus, upon receiving the mail, the aroma display can determine the scent using the event name as a key, and generates the determined scent. In the following, the aroma display used in the aroma scheduling system in accordance with the first embodiment will be described.

[Configuration]

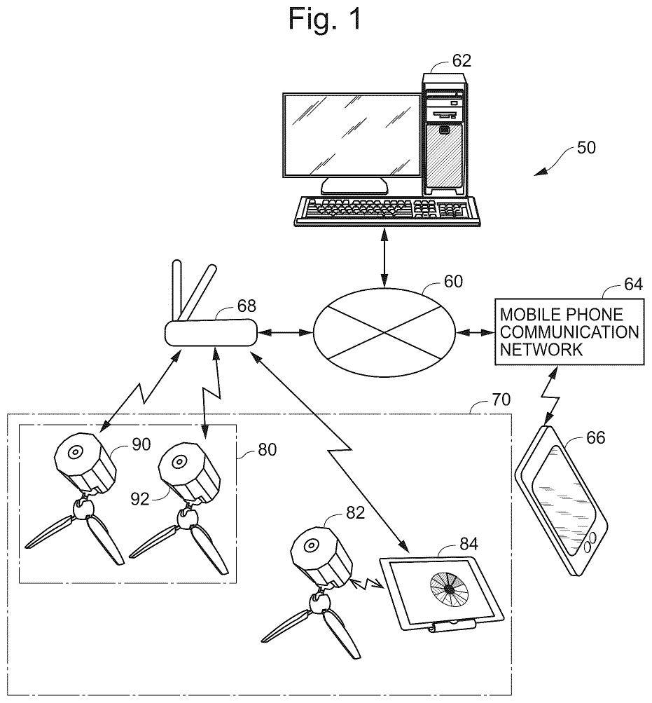

FIG. 1 schematically shows an overall configuration of an aroma scheduling system 50 using an aroma display in accordance with a first embodiment. Referring to FIG. 1, aroma scheduling system 50 includes: an existing scheduling server 62 connected to the Internet 60, for providing scheduling service to a plurality of clients; a smart phone 66 capable of communicating with scheduling server 62 through the Internet 60 and a portable telephone communication network 64, and capable of using the scheduling service of scheduling server 62; and a business facility 70 capable of communication with the Internet 60 and other terminals thereon through a wireless router 68 and having a plurality of aroma displays 82, 90 and 92. Aroma displays 82, 90 and 92 each allow loading of a plurality of (for example, six) aroma cartridges and emit scent from any of these aroma cartridges, like the one disclosed in Patent Literature 1.

In the building of business facility 70, there is an office 80, and at prescribed positions in office 80, aroma displays 90 and 92 are installed, which aroma displays are capable of receiving mails from scheduling server 62 through wireless communication with wireless router 68. Further, in the building of business facility 70, there are provided aroma display 82 and a panel-type computer 84 for controlling aroma display 82. Aroma display 82 and panel-type computer 84 are capable of wireless communication with each other, and panel-type computer 84 is further capable of communicating with scheduling server 62 through wireless router 68. Though only one aroma display 82 is shown as an aroma display capable of communicating with panel-type computer 84 in FIG. 1, generally, aroma display of a same type, or a plurality of different types communicate with panel-type computer 84 and are managed by panel-type computer 84.

It is noted that aroma displays 90 and 92 and panel-type computer 84 have their mail addresses, and respectively receive event notification mails from scheduling server 62 through a mail server, not shown.

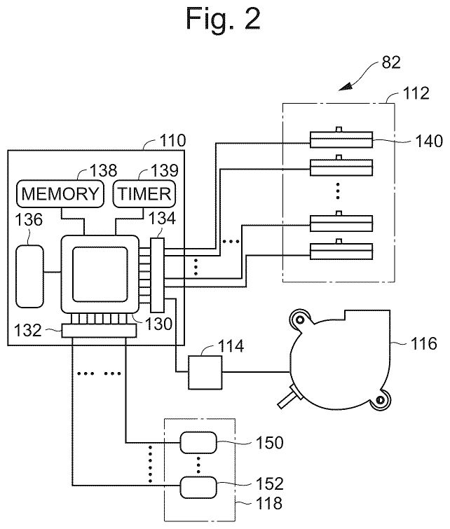

Referring to FIG. 2, aroma displays 82, 90 and 92 each include: a micro-blower group 112 including a plurality of micro-blowers 140 provided corresponding to respective cartridges to cause emission of scents from a plurality of aroma cartridges, not shown; a sirocco fan 116 for emitting scent-free air to dissipate any scent; a driving circuit board 114 for driving sirocco fan 116 in accordance with a condition designated by an externally applied command; a control board 110 capable of individually driving micro-blowers 140 included in the micro-blower group 112 and sirocco fan 116; and an NFC chip group 118 including a plurality of NFC chips 150, . . . , 152, connected to control board 110, obtaining an identification number representing scent sealed in each aroma cartridge loaded on aroma display 82 through near field communication with an NFC (Near Field Communication) tag (not shown) attached to each cartridge.

Control board 110 includes: a control processor 130 capable of wireless communication with the outside, for controlling the micro-blower group 112 and sirocco fan 116 based on externally applied information and on information from the NFC chip group 118; a memory 138 and a timer 139, both connected to control processor 130; an input-output OF 134 for controlling each of the micro-blowers 140, . . . in the micro-blower group 112 in accordance with instructions from control processor 130; an input-output I/F 132 receiving scent identifiers of respective cartridges from NFC chips 150, . . . 152 in the NFC chip group 118 and inputting them to control processor 130; and a wireless communication unit 136 enabling wireless communication of control processor 130 with the outside, for example, with the panel-type computer 84 (see FIG. 1). In aroma displays 90 and 92 shown in FIG. 1, wireless communication unit 136 communicates with scheduling server 62 through wireless router 68.

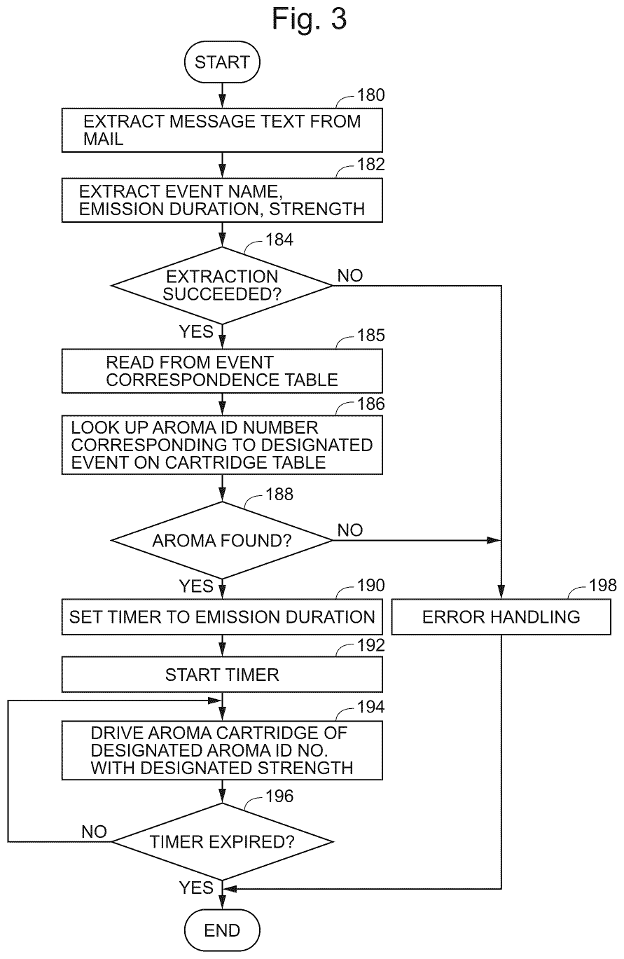

Memory 138 includes an ROM (Read-Only Memory) and an RAM (Random Access Memory). ROM stores programs for generating scents for events, which will be descried later with reference to FIG. 3, and a correspondence table (event correspondence table) indicating correspondence between event names and identification numbers (aroma identification number, or aroma ID) of scents to be generated at the events, to be used by the programs. Programs to be executed by control processor 130 is loaded to RAM, and the RAM is used as a work area by control processor 130. The aroma identification number is an identification number indicating the type of scent source sealed in the aroma cartridge. The aroma identification number is determined uniquely for each type of scent. Each aroma cartridge has an NFC tag (not shown) attached, which tag stores the aroma identification number identifying the scent source sealed in the aroma cartridge. Each aroma display obtains the aroma identification number of each aroma cartridge by near field communication with these NFC tags, and holds an aroma identification number table, as will be described later.

Aroma displays 90 and 92 shown in FIG. 1 receive an event notification directly, though naturally through a mail server, from scheduling server 62 and operate accordingly. In contrast, aroma display 82 does not directly receive an event notification from scheduling server 62. Panel-type computer 84 receives an event notification from scheduling server 62, interprets the notification and controls aroma display 82 and the like connected to panel-type computer 84. It is noted, however, that the programs executed by aroma displays 82, 90 and 92 have the same structure, as will be described later.

In the following description, when an event is to be registered with scheduling server 62, by way of example, one operates a smart phone 66 to enter a prescribed event name, start time and end time of the event, and a destination address of a notification e-mail, to be registered in scheduling server 62. Following the event name, one may add, with commas, duration and strength of scent emission from the aroma display, though these are optional and not essential.

FIG. 3 shows a control structure of a program causing aroma display 82, 90 or the like to generate a prescribed scent upon receiving an event notification, stored in memory 138 shown in FIG. 2. Referring to FIG. 3, this program is invoked in response to reception of a mail, which is an event notification, by control processor 130 shown in FIG. 2, through wireless communication unit 136. This program includes: a step 180 of extracting message text from the received mail; a step 182 of extracting an event name, emission duration (if any), and emission strength (if any) from the text extracted at step 180; and a step 184 of determining whether or not an event name has been successfully extracted at step 182, and branching the control flow depending on the result. Here, whether an event name has been successfully extracted or not is determined depending on whether or not the extracted character sequence is found in the event correspondence table pre-stored in memory 138 shown in FIG. 2.

Extraction of a plurality of elements separated by delimiters, such as commas, from mail texts can be realized easily by a program instruction and the like. If there are two (only two) elements after separation, the first is the event name and the second is the duration of emission. The duration of emission is a numeral, counted by seconds. If there are three elements, the first and the second are the event name and the duration as in the case of two elements, and the third indicates the strength of emission. In the present embodiment, the strength of emission is also given by a numeral. If the second and the third character sequences are absent or if the second and third character sequences are not a date or a numeral, these are ignored and prescribed default values are used as the duration and the strength of emission.

The program further includes: a steps 185, which is executed if the determination at step 184 is in the positive, that is, if at least a correct event name is extracted from the mail text, of reading an aroma identification number corresponding to the designated event name from the event correspondence table; a step 186 of looking up an aroma cartridge having the corresponding aroma identification number on the cartridge table stored in memory 138 shown in FIG. 2; and a step 188 of determining whether or not a cartridge having the corresponding identification number is found at step 186, and branching the control flow depending on the result.

The program further includes: a step 190, which is executed if the determination at step 188 is in the positive, of setting timer 139 shown in FIG. 2 to the emission duration extracted from the mail text at step 182 (if not extracted, default emission duration); a step 192, following step 190, of starting measurement of timer 139; a step 194 of driving the aroma cartridge having the designated aroma identification number with the strength extracted from the mail text at step 182 (if not extracted, default strength) and thereby emitting a scent; and a step 196 of determining whether the timer started at step 192 has expired or not, and if the timer has not expired, returning the control flow to step 194, and if the timer has expired, ending execution of this program.

The program further includes: a step 198 executed if the determination at step 184 is in the negative, or if the determination at step 188 is in the negative, of performing a predetermined error handling process (for example, sending an error message to a prescribed address, writing details of the error in a log file etc.), stopping generation of a scent if emission of a scent is in progress, and ending execution of the program.

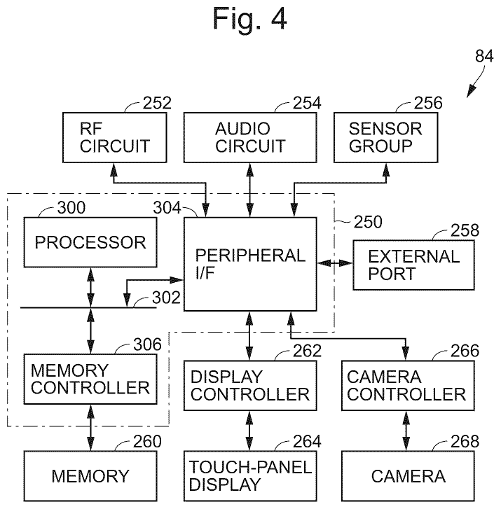

FIG. 4 is a block diagram schematically showing a hardware configuration of panel-type computer 84 shown in FIG. 1. Referring to FIG. 4, panel-type computer 84 includes a semiconductor integrated circuit 250 having a processor 300, a peripheral OF 304 and a memory controller 306 mounted thereon. Processor 300, peripheral OF 304 and memory controller 306 are capable of communicating with each other through a bus 302.

A memory 260 is connected to memory controller 306 of semiconductor integrated circuit 250. In the present embodiment, memory 260 includes a combination of a volatile memory and a non-volatile memory. The non-volatile memory includes a flash memory, which stores, for example, a basic program for operating processor 300, an application program and so on. The volatile memory is used as a working memory and as a main memory during execution of a program.

Panel-type computer 84 further includes: an RF circuit 252 capable of wireless communication with processor 300 through peripheral OF 304; an audio circuit 254; a sensor group 256; a display controller 262 and a camera controller 266; and an external port 258 connected to semiconductor integrated circuit 250, allowing connection of a portable memory, a portable hard disk and the like, not shown. A touch-panel display 264 is further connected to display controller 262. Similarly, a camera 268 is connected to camera controller 266.

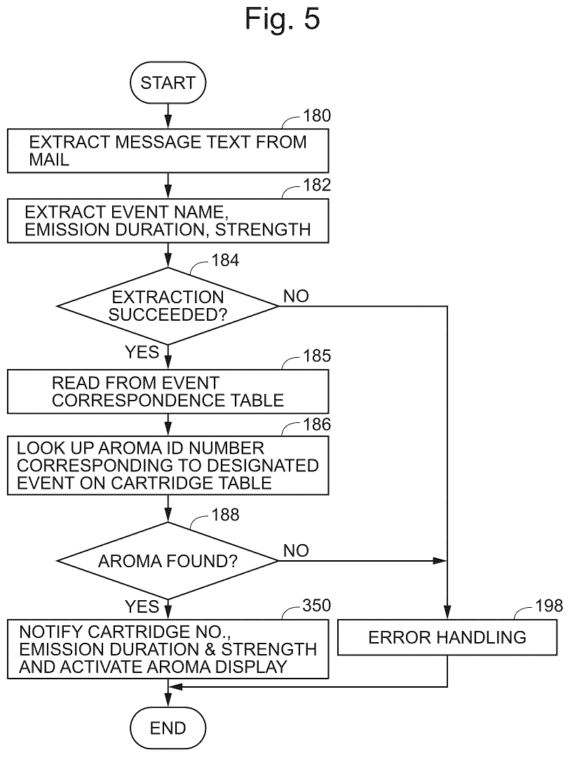

FIG. 5 is a flowchart showing a control structure of a program for controlling aroma display 82 in response to a message from scheduling server 62, stored in the non-volatile memory in memory 260 shown in FIG. 4. The program of which control structure is shown in FIG. 5 is invokedin response to reception of a mail from scheduling server 62.

Referring to FIG. 5, the program includes: a step 180 of extracting message text from the received mail; and a step 182 of extracting, from the message text extracted at step 180, event name, emission duration and information representing emission strength. At this step also, as at step 182 shown in FIG. 3, in the absence of emission duration and information representing emission strength, default values are used, respectively.

The program further includes: a step 184 of branching the control flow depending on whether an event name has been successfully extracted or not at step 182; a step 185, executed if the determination at step 184 is in the positive, of reading an aroma identification number corresponding to the designated event from the event correspondence table stored in memory 260; and a step 186 of looking up a cartridge table storing the aroma identification numbers from NFC tags attached to the cartridges loaded in aroma display 82 and loaded positions of respective cartridges, for the aroma cartridge having the read aroma identification number. The cartridge table is received beforehand by panel-type computer 84 from aroma display 82, and stored in memory 260 (see FIG. 4). The determination at step 184 is done by finding a record having the extracted event name on an event table prepared in advance storing event names tied to aroma identification numbers of scents to be emitted at the events. If the record is found on the table, the identification number of the scent to be generated at that event is simultaneously read.

The program further includes: a step 188 of determining whether or not the cartridge number of aroma cartridge having the identification number designated at step 186 is on the cartridge table, and branching the control flow depending on the result; a step 350, executed if the determination at step 188 is in the positive, of activating aroma display 82 to emit scented air from the aroma cartridge of the cartridge number read at step 186 for the designated duration with designated strength, and ending the process; and a step 198, executed if the determination at step 184 is in the negative, that is, if the event included in the received message is not found on the event correspondence table, or if the determination at step 188 is in the negative, that is, if the aroma cartridge having the aroma identification number read at step 185 is not on the cartridge table, of performing a prescribed error handling process and ending the process.

[Operation]

The scheduling system 50 described above operates as follows. Referring to FIG. 1, a user registers an event with scheduling server 62 by using, for example, smart phone 66. The aroma to be generated by aroma display 82 or the like is determined in advance, and in the form of an event correspondence table stored in aroma displays 82, 90 and 92 keyed to the event names. Registration of an event is done in the same manner as any common scheduling service. Specifically, a user registers event name, start time, end time, location and a destination of a mail to be sent at the start of the event. If the user wants to designate duration and strength of aroma emission, the duration and the strength are registered in a predetermined form (numeral) using commas as delimiters, following the event name.

Scheduling server 62 registers the event on a schedule table, not shown. Thereafter, at a prescribed time interval, a process of checking the schedule table iteratively runs to see whether or not there is an event immediately following a registered event start time, that is, whether or not there is an event of which start time is substantially the current time. When such an event is found, scheduling server 62 transmits a mail including the event name, the emission duration and strength, if any, separated by commas, to the mail destination designated for the event. Here, it is assumed that aroma displays 90, 92 and panel-type computer 84 use the same mail address and check with a relatively short time period whether a mail has been received.

By way of example, when aroma display 90 receives this mail, aroma display 90 operates as follows. Referring to FIG. 3, at step 180, a message text is extracted from the mail, and at step 182, the event name and the emission duration and strength, if any, are extracted from the message text. At step 184, whether or not this event name is on the event correspondence table is determined. If the event name is found, the determination is in the positive, and at step 185, the aroma identification number corresponding to the event is read from the event correspondence table. If the event name cannot be found on the event correspondence table, the determination is in the negative, so that a prescribed error handling process is done at step 198 and the process ends without emitting any scent.

At step 186, it is determined whether or not the cartridge having the aroma identification number read from the event table at step 185 is found on the cartridge table of aroma display 90. If such an aroma cartridge is not found on the cartridge table, the designated scent cannot be emitted. Therefore, control proceeds to step 198, where the error handling process is done, and the program ends. If such an aroma cartridge is found on the cartridge table, the scent can be generated by aroma display 90. Therefore, at step 190, the timer is set to the emission duration and at step 192, the timer is started. At the following step 194, the aroma cartridge of the designated identification number is driven with the designated aroma strength. Specifically, at step 194, a signal, which is an alternate signal of a prescribed signal length, of which amplitude is determined by the designated strength, is transmitted to the micro-blower of the cartridge corresponding to the designated identification number. When the output of the alternate signal for a prescribed time period ends, it is determined whether or not the timer has expired at step 196. If the timer has not expired, step 194 is repeated. In this manner, the scent of the designated identification number is emitted with the designated strength continuously for the emission duration, to the outside from the cartridge having the NFC tag of the identification number. When the timer expires, execution of the program ends.

If aroma displays 90 and 92 shown in FIG. 1 are set to receive mails to the same mail address and have the aroma cartridges of the same identification number, it follows that the same scent is emitted both from aroma displays 90 and 92 substantially at the same time with the same strength. Therefore, by installing a plurality of aroma displays such as aroma displays 90 and 92 in a limited space such as office 80, it becomes possible to spread a scent in the whole space.

On the other hand, different from aroma displays 90 and 92, in the case of aroma display 82, panel-type computer 84 is interposed. Here, it is assumed that the same mail address as aroma displays 90 and 92 is allotted to panel-type computer 84. Then, referring to FIG. 5, at step 180, panel-type computer 84 extracts a message text from the received mail, and at step 182, extracts information related to the event name and, if any, emission duration and emission strength, from the message text. At step 184, it is determined whether or not an event name has been successfully extracted. Specifically, if the extracted event name is on the event table held by panel-type computer 84, it is determined that the extraction was successful, and if not, it is determined to be a failure. When extraction of an event name fails, an error handling process is done at step 198, and the program ends. Therefore, no scent is emitted.

When an event name is successfully extracted, the aroma identification number corresponding to the event name is read from the event correspondence table at step 185, and the aroma cartridge having the aroma identification number is looked up on the cartridge table of aroma display 82 (step 186). Specifically, panel-type computer 84 looks up the cartridge table obtained by communication with aroma display 82 and stored in advance, for the aroma cartridge having the designated identification number. At the following step 188, it is determined whether or not such an aroma cartridge is found. If such an aroma cartridge is not found, the designated scent cannot be emitted and, therefore, determination at step 188 is in the negative, an error handling process is done at step 198, and the program ends without emitting any scent.

If it is determined at step 188 that the cartridge having the designated aroma identification number is loaded on aroma display 82, at step 350, a command is issued to aroma display 82 so that the micro-blower driving the cartridge among the micro-blower group 112 shown in FIG. 2 is driven to emit the scent for the designated emission duration with the designated strength. As a result, aroma display 82 is activated in response to the command, and causes the designated micro-blower to emit the scent for the designated duration with the designated strength. Panel-type computer 84 executes such a process on every aroma display which is under control of panel-type computer 84.

[Effects]

As described above, in the aroma scheduling system 50 in accordance with the present embodiment, simply by effectively using a common scheduling service and by scheduling common registration related to an event, it becomes possible to emit a scent allotted to the event at a desired location and at a desired time. For example, when a meeting takes place or one mediates in a certain room, an aroma or scent corresponding to the contents of the event such as the meeting or the mediation can be emitted from the aroma display positioned at that location, simply by designating the event and the location. If there is a work break at prescribed time at a workplace, it is possible to emit refreshing aroma when the break starts. As a result, there is an effect that aromas or scents suitable for various events can be enjoyed simply by an operation of event scheduling.

### Second Embodiment

[Configuration]

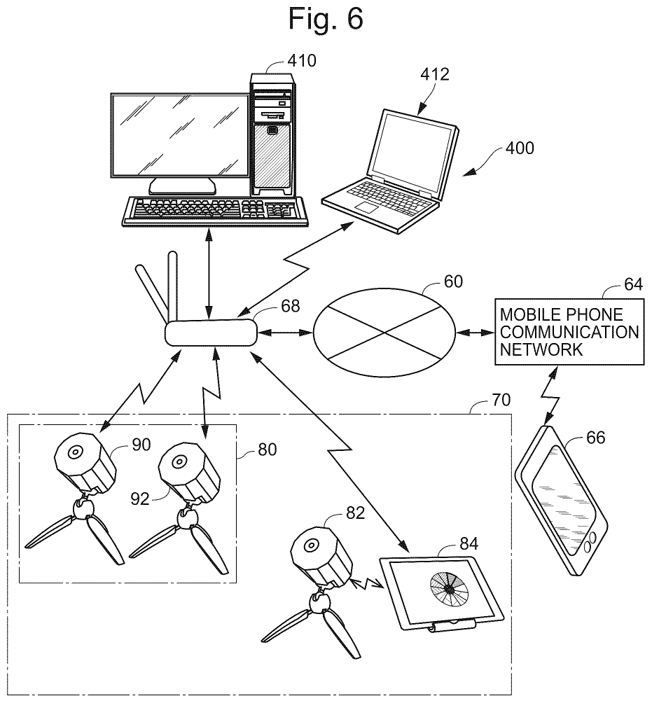

In the first embodiment described above, generation of a scent is controlled by using an external scheduling service. If such a scheduling service is available within a company, a system more flexible than the first embodiment can be built. The second embodiment is directed to such an in-company scheduling system 400 (FIG. 6).

Referring to FIG. 6, as compared with the aroma scheduling system 50 shown in FIG. 1, in-company scheduling system 400 is different from the aroma scheduling system 50 shown in FIG. 1 in that it includes an in-company scheduling server 410 connected to an in-company LAN (Local Area Network) of the business facility 70 through a wireless router 68 in place of scheduling server 62 of FIG. 1, and that it includes a computer 412, similarly connectable to the in-company LAN through wireless router 68, for setting in-company scheduling server 410. Except for these points, the hardware configuration is the same as that of aroma scheduling system 50.

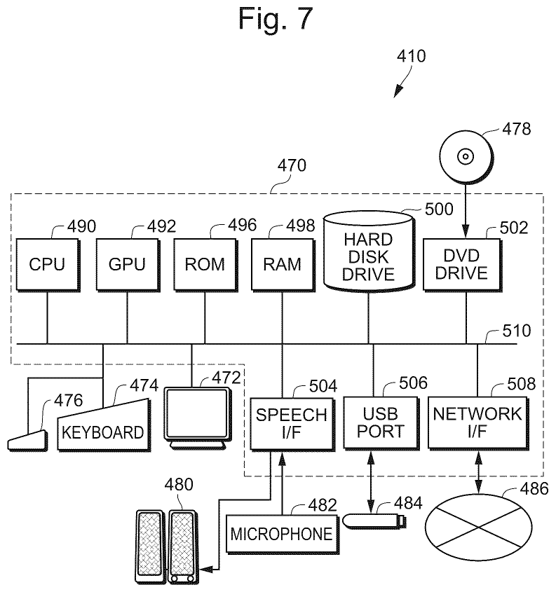

Referring to FIG. 7, in-company scheduling server 410 includes: a computer 470 having a DVD drive 502 to which a DVD (Digital Versatile Disk) 478 can be loaded, and a keyboard 474, a mouse 476 and a monitor 472, all connected to computer 470, enabling interaction with the user. These are merely examples of devices allowing user interaction, and any common hardware and software available for user interaction (such as touch-panel, speech input, pointing device and so on) may be used.

Computer 470 includes, in addition to DVD drive 502, a CPU 490, a GPU 492, a bus 510 connected to CPU 490, GPU 492 and DVD drive 502, a ROM 496 connected to bus 510, storing a boot-up program and the like of the computer 470, a RAM 498 connected to bus 510, storing instructions forming a program, a system program and work data, and an HDD (Hard Disk Drive) 500, which is a non-volatile memory connected to bus 510. HDD 500 is to store programs to be executed by CPU 490 and GPU 492 and storing data and the like used by the programs executed by CPU 490 and GPU 492 even when computer 470 is turned off. Computer 470 further includes a network OF 508 providing connection to in-company LAN 486 allowing communication with other terminals, a USB port 506 to which a USB memory 484 is detachably attached, providing communication between USB memory 484 and various units in computer 470, a speech OF 504 connected to bus 510, and a speaker 480 and a microphone 482 connected to speech OF 504.

Basic configuration of computer 412 shown in FIG. 6 is the same as that shown in FIG. 7.

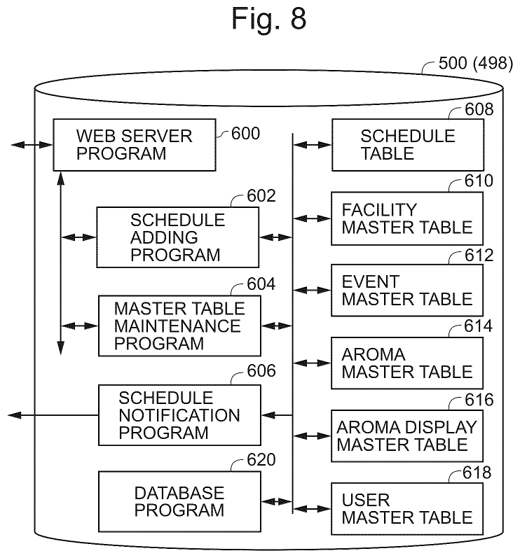

FIG. 8 schematically shows configurations of a program and various tables of a database, used by in-company scheduling server 410 in accordance with the second embodiment, stored in HDD 500 or RAM 498. Referring to FIG. 8, the program executed by in-company scheduling server 410 includes: a web server program 600 providing an interface of scheduling service; a schedule adding program 602 executed for registering an event as a schedule, among the scheduling services; a master table maintenance program 604 maintaining various master tables among various tables as will be described later; a schedule notifying program 606 performing a process of transmitting a schedule notification as a message to a prescribed contact address for an event of which start time has reached, among the scheduled events; and a database program 620 maintaining the database as will be described later. Of these programs, web server program 600 and database program 620 are loaded and resident in the memory. Other programs are typically stored in HDD 500 except when executed, and loaded to the memory when executed. If there is a room in the memory, these are resident in the memory and when memory capacity runs out, these are deleted from the memory.

The database for scheduling service used in the present embodiment includes: a schedule table 608 for holding information on registered schedules; a facility master table 610 for storing in advance locations where events may possibly take place; an event master table 612 for storing in advance events that may possibly be scheduled; an aroma master table 614 for storing information on various scents that may possibly be emitted at various events; an aroma display master table 616 for storing information on aroma displays that exist in the areas covered by in-company scheduling server 410; and a user master table 618 for storing information on users of the scheduling service provided by in-company scheduling server 410. These tables are basically resident in the memory when the database is in use, while they are written back to HDD 500 as needed if memory capacity runs out.

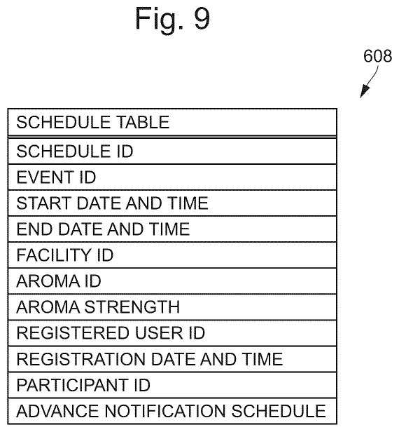

FIG. 9 shows contents of schedule table 608 shown in FIG. 8. Referring to FIG. 9, each record of schedule table 608 includes: a unique schedule ID; an event ID representing event type (event name); start date and time and end date and time of the event; a facility ID indicating the facility where the event takes place; an aroma identification number (aroma ID) indicating the scent to be generated at the event; aroma strength designating the strength of the scent to be generated a registrant ID indicating the user who registered the corresponding schedule; registration date and time indicating the date and time when the corresponding schedule is registered; a participant ID as an ID of the user who participates in the event; and a pre-notification schedule for notifying the participant or participants at a prescribed date and time before the start of event.

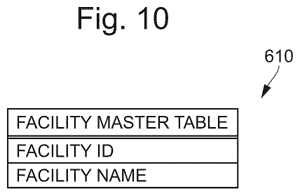

FIG. 10 shows a configuration of each record in facility master table 610 shown in FIG. 8. Referring to FIG. 10, facility master table 610 includes a unique facility ID and a corresponding facility name.

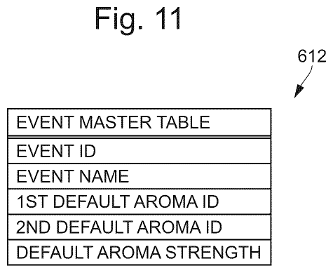

FIG. 11 shows a configuration of each record in event master table 612 shown in FIG. 8. Referring to FIG. 11, each record of event master table 612 includes a unique event ID, an event name, a default aroma ID as the identification number of the default aroma to be generated at the event, and a default aroma strength.

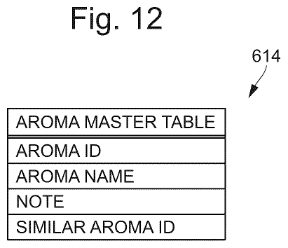

FIG. 12 shows a configuration of each record in aroma master table 614 shown in FIG. 8. Referring to FIG. 12, each record of aroma master table 614 includes an aroma ID as the aroma identification number, a name of the aroma or scent, and a note. The note is a reference for the user to help select the scents.

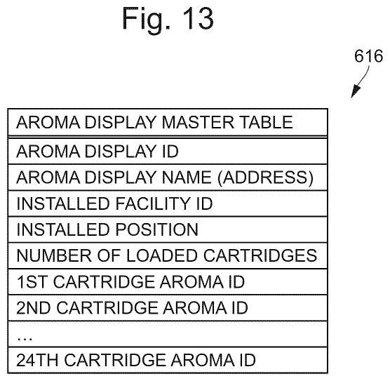

FIG. 13 shows a configuration of each record in aroma display master table 616 shown in FIG. 8. Referring to FIG. 13, aroma display master table 616 includes; a unique aroma display ID; an aroma display name; a facility ID as the identification number of the facility where the aroma display is installed; a location in the facility where the aroma display is placed; the number of cartridges held by the aroma display; and 1st to 24th aroma IDs. In the present embodiment, the number of aroma cartridges loadable to each aroma display is at most 24, though it depends on the aroma display. Further, the aroma display name is also used as an address to access the aroma display in the network.

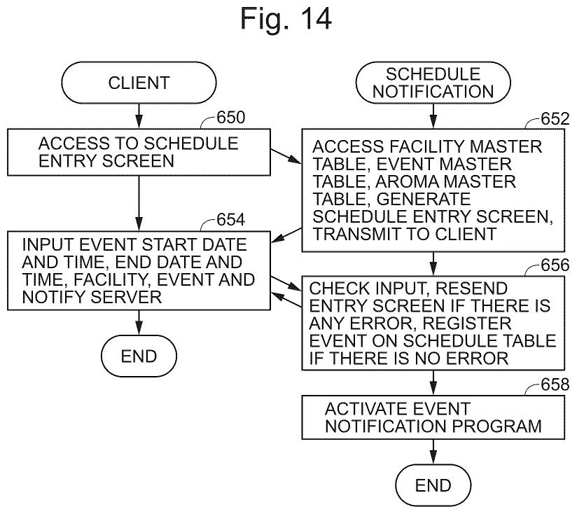

FIG. 14 is a sequence diagram between computer 412 and in-company scheduling server 410 when a client (for example, computer 412) accesses in-company scheduling server 410 for registering a schedule. Referring to FIG. 14, at step 650, computer 412 accesses a schedule entry screen of scheduling server 410. In response to this access, at step 652, in-company scheduling server 410 accesses facility master table 610, event master table 612, and aroma master table 614, generates a schedule entry screen and transmits it to computer 412. When computer 412 accesses in-company scheduling server 410, it is necessary to log-in the in-company scheduling server 410 by using the user ID of the user operating computer 412. Whether the user is authorized to access, or authorized to set a schedule is determined depending on whether the user is registered on the user master table 618 and on the authority allotted to the user.

At step 654, computer 412 displays the schedule entry screen transmitted from in-company scheduling server 410, allowing the user to enter the event name, event start date and time, event end date and time and the facility ID where the event takes place. When inputs are all entered, computer 412 transmits the input information to in-company scheduling server 410. Here, for entering the inputs, it is preferred that by using the information obtained by accessing facility master table 610, event master table 612 and aroma master table 614 etc. at step 652, a drop box, for example, listing the event name and the event ID, the facility name and the facility ID, the aroma name and the aroma ID is shown to allow easy input of the event. From computer 412 to in-company scheduling server 410, event ID, facility ID and aroma ID are transmitted, in addition to the event start time and end time.

In response to this communication, in-company scheduling server 410 checks this input at step 656 and if there is any error, resends the entry screen to computer 412 with an error message. If there is no error, information related to the event is registered with schedule table 608. Thereafter, the event notification program is activated at step 658 and the process ends.

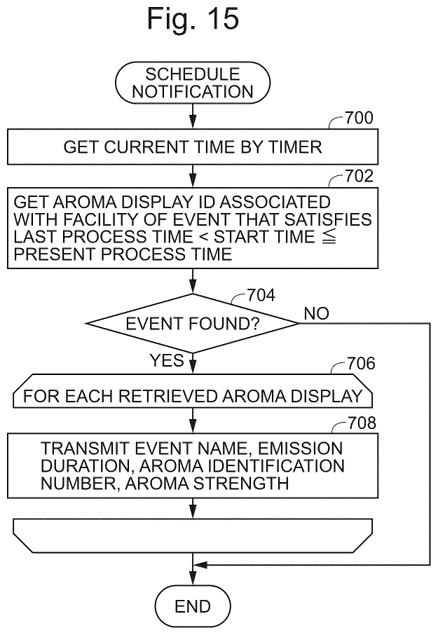

FIG. 15 shows a control structure of the event notification program. The event notification program is to find, from the events registered with schedule table 608, any event of which start time has just arrived, that is, any event of which start time is substantially the same as the current time, and if any, notifies the aroma display placed at the facility registered for the event of the event name, aroma emission duration, aroma ID of the scent to be emitted, and aroma strength. Basically, this program is repeated with a prescribed time interval and, in addition, it is executed immediately after any event is registered as indicated by step 658 of FIG. 14. In the present embodiment, the scent emission duration is the event duration time calculated from the read start and end times of the event.

Referring to FIG. 15, the event notification program includes: a step 700 of obtaining the current time from the timer; a step 702 of searching for any event of which start time is between the last processing time (the time this program was last executed) and the present time on schedule table 608, and reading from aroma display master table 616 a record of that aroma display which has the facility ID stored in the record of such an event; and a step 704, following the result of step 702, of determining whether or not a corresponding event has been found and if not, ending execution of the program.

The program further includes: a step 706, executed if the determination at step 704 is in the positive, of executing step 708 for each aroma display retrieved at step 702, and ending execution of the program.

At step 708, in-company scheduling server 410 transmits the event name, the aroma emission duration, the aroma ID and the aroma emission strength to the aroma display as the object of processing.

On the other hand, among the aroma display IDs searched at step 702, an ID of a computer such as panel-type computer 84 shown in FIG. 1 that controls one or more aroma displays collectively is included. This is to facilitate, when a plurality of aroma displays are arranged in one room, control of aroma displays room by room, or control of a plurality of aroma displays to generate a uniform aroma or scent at various locations in the facility.

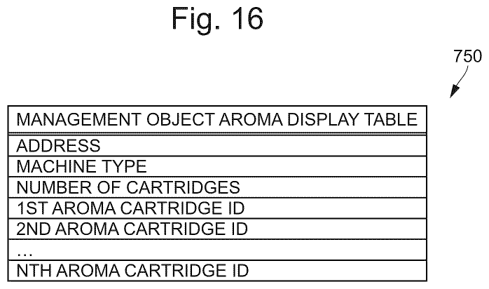

FIG. 16 shows a configuration of each record of object aroma display table 750 for controlling aroma displays, held in such a computer. Referring to FIG. 16, each record of object aroma display table 750 includes: aroma display name (address) of the aroma display; name of its type; the number of aroma cartridges loaded to the aroma display; and aroma IDs of the 1st to 24th aroma cartridges. Here again, it is assumed that at most 24 aroma cartridges are loadable to each aroma display.

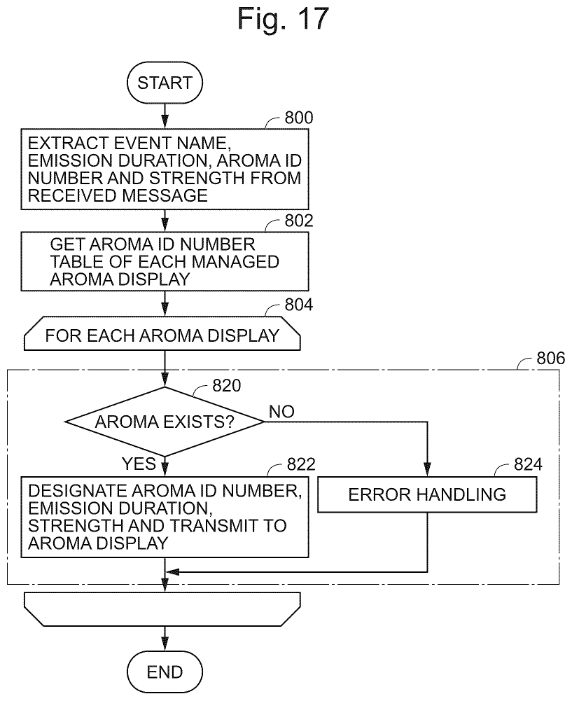

FIG. 17 is a flowchart showing a control structure of a program executed by a computer (management computer) that collectively manages one or more aroma displays. Referring to FIG. 17, the program includes: a step 800 of extracting the event name, the emission duration, the aroma identification number and the emission strength from the message received from in-company scheduling server 410; a step 802 of obtaining the aroma identification number table held by each aroma display as an object of management; and a step 804 of executing a step 806 on each aroma display and ending execution of the program. Each aroma display obtains, by near field communication between the NFC tags of loaded aroma cartridges and NFC chips, aroma identification numbers of these aroma cartridges and holds them on the aroma identification table.

Step 806 includes: a step 820 of determining whether or not the aroma cartridge having the aroma ID designated by in-company scheduling server 410 is loaded on the aroma display as the object of processing, and branching the control flow depending on the result; a step 822, executed if the determination at step 820 is in the positive, of transmitting a command to generate a scent to the aroma display as the object of processing, designating the aroma ID of the scent to be generated, the emission duration and the emission strength, and ending execution of step 806; and a step 824, executed if the determination at step 820 is in the negative, of executing a prescribed error handling process, and ending execution of step 806.

The process of step 802 is done every time since there is a possibility that the aroma cartridges loaded to each aroma display is changed as needed.

[Operation]

In the present embodiment, the event name, the emission duration, the aroma ID and the aroma strength are transmitted directly from in-company scheduling server 410 to each object aroma display 90, 92 or to the management computer such as panel-type computer 84, not through a mail. Aroma display 90 or 92, which is not an object of management by the panel-type computer 84, operates in the same manner as in the first embodiment except that a message is directly received from in-company scheduling server 410, rather than from a mail received by accessing a mail server.

When a panel-type computer 84 is interposed, the panel-type computer 84 and the aroma display 82 under the control of panel-type computer 84 operate as follows.

The panel-type computer 84 extracts, from the message received from in-company scheduling server 410, the event name, the emission duration, the aroma ID and the emission strength (step 800). Further, the panel-type computer 84 obtains the aroma ID table of each aroma display it managest (step 802). At step 806, the panel-type computer 84 executes step 806 on each aroma display and ends execution of the program.

At step 806, the panel-type computer 84 determines whether or not the aroma cartridge having the aroma ID designated by in-company scheduling server 410 is loaded to the aroma display 82 to be processed, and branches the control flow depending on the result (step 820). If the aroma cartridge having the designated aroma ID is loaded to the aroma display 82 to be processed (if the determination at step 820 is in the positive), the panel-type computer 84 transmits a command to generate a scent to the aroma display 82 to be processed, designating the aroma ID of the scent to be emitted, the emission duration and the aroma strength, and ends execution of step 806 (step 822). If the aroma cartridge having the designated aroma ID is not loaded to the aroma display 82 to be processed (if the determination at step 820 is in the negative), the panel-type computer 84 executes a prescribed error handling process and ends execution of step 806 without transmitting any command to the aroma display 82 (step 824).

The operation of each aroma display managed by the panel-type computer 84 is the same as the first embodiment.

[Effects]

In the second embodiment, different from the first embodiment, a message is transmitted not by a mail but directly from the in-company scheduling server 410 to each aroma display or to the management computer. Therefore, as soon as an event start time arrives, an event notification for generating a scent is transmitted substantially without any delay, to each aroma display and to the management computer, so that a prescribed scent is emitted at each location without delay. It becomes unnecessary for each aroma display and the management computer to repetitively access the mail server, reducing load on the network.

When an aroma display is to be managed through a management computer, in order to simultaneously operate aroma displays placed in the same room or the like, what is necessary for in-company scheduling server 410 is simply to transmit a message only to the management computer. Therefore, management of aroma displays by in-company scheduling server 410 becomes simpler.

### Third Embodiment

[Configuration]

Both in the first and second embodiments above, a scent is generated only at the event start time. If duration of aroma emission is made longer, the aroma effect lasts longer. The scent source, however, will be used up faster, which is economically undesirable. Once a scent is emitted, it lasts for a while. Therefore, it is not preferable to make the duration longer.

On the other hand, it is sometimes desirable to maintain the aroma effect all through an event. If there is such a request, in the first and second embodiments, this effect can be attained by registering one same event a number of times, with the start time set at prescribed intervals. This procedure, however, is undesirable from the viewpoint of schedule management, since originally one event is registered a number of times.

Therefore, when registering a schedule with in-company scheduling server 410, for example, time interval of aroma emission may be registered separately from the schedule start time. Here, a field for time interval is made in schedule table 608 shown in FIG. 9. At step 702 of FIG. 15, in place of event start time, the object of comparison is set to be event start time+aroma emission time interval x i (i=0, 1, K where K is a natural number such that event start time+aroma emission time interval x K is smaller than the event end time, and that event start time+aroma emission time interval x (K+1) is larger than the event end time).

[Operation]

With this configuration, when the program of FIG. 15 is executed by in-company scheduling server 410, the determination at step 704 will be in the positive at every prescribed time from the event start time to the event end time, and the scent is emitted every time from the aroma display.

[Effect]

Therefore, according to this embodiment, an aroma is emitted at a prescribed time interval from the start to the end of an event and the aroma effect can be maintained.

### Fourth Embodiment

In any of the above-described embodiments, the device for schedule management and the device for emitting aroma are separate devices. The present invention, however, is not limited to such embodiments. The device for schedule management may be the same as the device for emitting aroma. Specifically, the aroma display 82 shown in FIG. 1 may have the schedule management function.

Here, the program of which control structure is shown in FIG. 15 and the program of which control structure is shown in FIG. 3 or FIG. 5 are operated separately in the aroma display, and messages may be exchanged therebetween. Alternatively, the program of which control structure is shown in FIG. 15 and the program of which control structure is shown in FIG. 3 or FIG. 5 may be integrated to be one program.

In this case, the schedule itself may be adopted to be registered with the aroma display only, or a schedule formed by another computer or the like may be downloaded to the aroma display.

By enabling aroma emission event scheduling on the aroma display, it becomes possible for each user to separately schedule his/her own aroma display. As a result, each user can enjoy his/her favorite scents whenever he/she likes and, therefore, it becomes possible to spend well-modulated work and daily life.

The tembodiments as have been described here are mere examples and should not be interpreted as restrictive. The scope of the present invention is determined by each of the claims with appropriate consideration of the written description of the embodiments and embraces modifications within the meaning of, and equivalent to, the languages in the claims.

### REFERENCE SIGNS LIST

50 aroma scheduling system
60 Internet
62 scheduling server
64 portable telephone communication network
66 smart phone
68 wireless router
70 business facility
80 office
82, 90, 92 aroma display
84 panel-type computer
110 control board
112 micro-blower group
114 driving circuit board
116 sirocco fan
118 NFC chip group
130 control processor
132, 134 input-output OF
136 wireless communication unit
138, 260 memory
139 timer
140 micro-blower
150, 152 NFC chip
250 semiconductor integrated circuit
252 RF circuit
254 audio circuit
256 sensor group
258 external port
262 display controller
264 touch-panel display
266 camera controller
268 camera
300 processor
304 peripheral OF
306 memory controller
400 in-company scheduling system
410 in-company scheduling server
486 in-company LAN
600 web server program
602 schedule adding program
604 master table maintenance program
606 schedule notifying program
608 schedule table
610 facility master table
612 event master table
614 aroma master table
616 aroma display master table
618 user master table
620 database program
750 object aroma display table

1. An aroma display, comprising:
a message receiving means for receiving an external message;
a correspondence storage means for storing correspondence between an event name of an event and aroma information related to a scent to be generated at the event;
an event notification extracting means for extracting an event notification including an event name from a message received by said message receiving means;
an aroma information reading means for reading aroma information corresponding to the event name from said correspondence storage means; and
an aroma emitting means for emitting a scent in accordance with the aroma information read by said aroma information reading means.

2. The aroma display according to claim 1, wherein
said aroma emitting means allows loading of a plurality of aroma cartridges each having an identification number indicative of its scent source, and is capable of emitting a scent from any designated aroma cartridge among the plurality of aroma cartridges;
said aroma information includes said identification number; and
said aroma emitting means includes
an identification number storage means for storing said identification number allotted to each of said plurality of aroma cartridges loaded to said aroma emitting means, and
a cartridge driving means for driving that aroma cartridge which has said identification number included in said aroma information among said plurality of aroma cartridges loaded to said aroma emitting means and thereby for generating a scent.

3. The aroma display according to claim 2, wherein
said event notification may include a duration of scent emission; and
in response to said event notification including information related to the duration of scent emission, said cartridge driving means drives said aroma cartridge for the duration from the start time of said event.

4. The aroma display according to claim 3, wherein
in response to said event notification not including the duration of scent emission, said cartridge driving means drives said aroma cartridge for a predetermined default time period from the start time of said event.

5. An aroma display control device, for controlling one or a plurality of aroma displays, comprising:
a communication destination information storage means for storing communication destination information enabling communication with said one or a plurality of aroma displays as communication destinations;
a message receiving means for receiving an external message;
a correspondence storage means for storing correspondence between an event name of an event and aroma information related to a scent to be generated at the event;
an event notification extracting means for extracting an event notification including an event name from a message received by said message receiving means;
an aroma reading means for reading aroma information corresponding to said event name from said correspondence storage means; and
a command transmitting means for transmitting a command to an aroma display as a communication destination stored in said communication destination information storage means so that a scent is emitted in accordance with the aroma information read by said aroma reading means.

6. The aroma display control device according to claim 5, wherein said command transmitting means includes a full transmission means for transmitting said command to each of said aroma displays of communication destination stored by said communication destination information storage means to generate a scent in accordance with the aroma information read by said aroma reading means.

7. The aroma display control device according to claim 5, wherein
said event notification may include information related to a duration of scent emission in an event indicated by said event notification; and
said command transmitting means includes a means, responsive to said event notification including said information related to the duration, for transmitting said command including said event name and said information related to the duration, to an aroma display of communication destination stored by said communication destination information storage means to generate a scent in accordance with the aroma information read by said aroma reading means.

8. A computer program causing a computer to function as the aroma display control device in accordance with claim 5.

9. An aroma scheduling server, comprising:
a timer;
a correspondence storage means for storing correspondence between an event name of an event and an identification number of a scent to be generated at the event;
an access information storage means storing access information to an aroma display;
a schedule information storage means, receiving from a user, inputs of an event name, start time and end time of the event, and location information specifying the location where the event takes place, for storing schedule information including the event name, the start time and the end time, and said location information;
an identification number reading means for reading, from said correspondence storage means, an identification number of a scent corresponding to said event name input by said user, and for adding it to said schedule information corresponding to said event name;
a schedule information reading means for detecting whether a piece of schedule information having substantially the same start time as the time indicated by the timer exists in the schedule information stored in said schedule information storage means, and for reading the detected piece of schedule information; and
an event notification transmitting means for transmitting an event notification including scent identification information, scent emission duration, and an event name determined in accordance with the read piece of schedule information to a destination determined by the access information included in the piece of schedule information read by said schedule information reading means; wherein
said schedule information storage means includes an emission-interval-added schedule information storage means, receiving, from a user, inputs of an event name, start time and end time of the event, and location information specifying the location where the event takes place and time interval for emitting a scent at the event, for storing schedule information including the event name, the start time and the end time, said location information and the emission time interval;
said scheduling server further comprising:
an aroma emission time calculating means for calculating aroma emission time at which said scent is to be emitted for said event based on the event start time and the emission time interval;
said schedule information reading means includes an aroma emission schedule information reading means for detecting whether a piece of schedule information having the emission time substantially the same as the time indicated by said timer exists in said schedule information stored in said emission-interval-added schedule information storage means, and for reading the detected piece of schedule information.

10. A computer program causing a computer to function as the aroma scheduling server according to claim 9.
